(19) United States
(12) Patent Application Publication
(10) Pub. No.: US 2007/0287852 A1
(43) Pub. Date: Dec. 13, 2007

(54) Method of Preparing Liquid Nitrate Esters

(76) Inventors: Jurgen Antes, Karlsruhe (DE); Dusan Boskovic, Karlsruhe (DE); Jurgen Haase, Waltrop (DE); Stefan Lobbecke, Karlsruhe (DE); Cornelius Ruloff, Leverkusen (DE); Tobias Turcke, Karlsruhe (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/589,579

(22) PCT Filed: Feb. 15, 2005
(86) PCT No.: PCT/EP05/01505

§ 371 Date: Jul. 26, 2007

(30) Foreign Application Priority Data
Feb. 16, 2004 (DE) 10 2004 007 706.1

(51) Int. Cl. C07C 201/02; C07C 203/06

(52) U.S. Cl. 558/480

(57) ABSTRACT

The invention relates to a method for the production of liquid nitrate esters, e.g. nitro-glycerine, wherein an alcohol is esterfied by means of nitrating acid and the reaction takes place in one or several microreactors.

The invention relates to a method of preparing liquid nitrate esters.

Liquid nitrate esters, for example glyceryl trinitrate (nitroglycerol), are obtained by reacting nitrating acid (a mixture of nitric acid, sulfuric acid and sulfur trioxide) with an alcohol, for example glycerol (cf. Winnacker & Küchler, “Chemische Technologie” (Chemical Technology), Volume 7, 1986, pages 359 to 402). The preparation and handling of nitroglycerol involves dangers.

It is therefore the object of the invention to overcome the disadvantages of the prior art and, in particular, to provide a method of preparing liquid nitrate esters, such as nitroglycerol, that is safer than the methods known hitherto.

The object is achieved by a method of preparing liquid nitrate esters, which method has the features of the main claim. Preferred refinements of the method according to the invention are to be found in the subclaims.

Microreactors or micromixers are extremely miniaturized tubular reactors having channel dimensions in the submillimetre range or volumes in the submillilitre range and are known per se. Descriptions are found, for example, in:

V. Hessel and H. Löwe, “Mikroverfahrenstechnik: Komponenten, Anlagenkonzeption, Anwenderakzeptanz”, (Microprocess technology: components, equipment design, user acceptance), Chem. Ing. Techn. 74, 2002, pages 17-30, 185-207 and 381-400.

J. R. Burns and C. Ramshaw, C., “A Microreactor for the Nitration of Benzene and Toluene”, in: Proceed. 4^{th} Int. Conference on Microreaction Technology (IMRET 4), 2000, Atlanta, USA.

S. Löbbecke et al., “The Potential of Microreactors for the Synthesis of Energetic Materials”, 31^{st} Int. Annu. Conf. ICT: Energetic Materials—Analysis, Diagnostics and Testing, 33, 27-30 June 2000, Karlsruhe, Germany.

Surprisingly, it was found that the esterification of alcohols can be performed by means of nitrating acid in a microreactor and the method found has the following advantages, not least for safety reasons.
A lower hold-up during the esterification reaction and the working-up steps in the equipment parts reduces the dangerous amounts of substance to be handled to the g-quantity range.
The thermal explosion risk is markedly reduced since, as a result of the very large surface-to-volume ratio in the miniaturized reactor structures, local overheating (hotspots) can be avoided with certainty.
The very short startup and shutdown times of the process reduce product flows that have to be disposed of or worked up in another way and that are not according to specification.
The short startup and shutdown times furthermore reduce the potential danger since the greatest process fluctuations occur (may occur) precisely in these process phases in which a steady state does not yet exist.
Short reaction and dwell times generally reduce the safety risk.
As a result of the continuous mode of operation, staffing can in principle be reduced.
The method results in reaction acceleration and, consequently, in shortened reaction times because markedly higher reaction temperatures (30 to 50° C. instead of the otherwise usual 25 to 30° C.) can be achieved without increasing the safety risk in the process.
The method ensures a completely isothermal mode of operation.
The wastewater flows can be significantly reduced by up to 75%.
The method according to the invention offers the possibility of scalably and economically producing both small and large product quantities since hardly any nitrate ester quantities produced are lost during the startup and shutdown of the equipment. The product quantities may, if necessary, be flexibly adapted.

Basically, microreactors in which fluid flows are mixed with one another are suitable for the method according to the invention. Microreactors that employ the split-and-recombine principle or microreactors that employ the multilamination principle or microreactors that bring the fluid flows into contact simply in a T-piece type of configuration may be mentioned here by way of example.

In a microreactor employing the split-and-recombine principle, fluid flows are split and brought together again after traversing different path sections. Repeating this flow configuration several times, for example microchannels repeatedly disposed in parallel, results in efficient mixing of the liquid flows. The internal channel diameters of the microchannel structures of such microreactors are approximately 50 to 3000 μm. The length of the parallel microchannel structures may vary between 1 and 50 mm, preferably between 15 and 20 mm.

In a microreactor employing the multilamination priciple, the individual fluid flows are first divided up into parallel lamellar flows before they are alternately combined and consequently mixed with the second multilaminated fluid flow. The internal channel diameters of the microchannel structures of such microreactors are approximately 50 to 3000 μm. The length of the parallel microchannel structures may vary between 1 and 50 mm, preferably between 15 and 20 mm.

The internal channel diameters of the microreactors may vary between 50 to 3000 μm. Preferably, internal channel diameters of 100 to 1000 μm and, very particularly preferably, of 200 to 300 μm are used.

Preferably, a laminar flow of the liquids is employed in the reaction in the microreactor, the Reynolds number being particularly preferably below 1000.

In the method, microreactors are used that ideally contain microstructured passive mixing structures. However, simple T-mixers or Y-mixers having comparable internal channel dimensions can also be used.

Preferably, microreactors using glass or silicon as material are used. In addition, reactors using materials of metal, ceramic or enamel can also be used.

According to the invention, provision may be made, in addition, to connect a plurality of identical or different microreactors in series downstream of one another (microreactor systems).

According to the invention, provision may further be made that, after leaving the microreactor, the reaction mixture flows through a temperature-controlled dwell section, for example a Teflon capillary. In that case, the microreactor and the dwell section form a microreactor system. The length of the dwell section may be varied relatively freely within wide limits, for example it may be 20 to 100 cm, preferably 40 to 80 cm, particularly preferably 50 cm. The internal diameter of this capillary may be 500 to 3000 μm, preferably 800 μm.

The chosen internal channel diameter of the microreactors/microreactor systems establishes a very large surface-to-volume ratio. This achieves a preferred isothermal mode of operation.

Preferably, monohydric or polyhydric alcohols are used as alcohols. Very particularly preferably, glycerol is used as alcohol.

Preferably, a mixture of concentrated sulfuric acid and concentrated nitric acid in a weight ratio of 0.8:1 to 1.2:1 is used as nitrating acid. In this connection, the concentrated sulfuric acid may contain oleum.

Preferably, the concentrated sulfuric acid contains up to 10 wt % oleum, particularly preferably 2 to 6 wt %. The reaction may, however, also be performed without oleum.

The nitrate esters produced by the method according to the invention may be mono-, di- or polynitrate esters. Particularly preferred is trinitroglycerol or glycyl dinitrate ester.

In the preparation of nitroglycerol, the molar ratio of HNO₃ to glycerol is preferably 3:1 to 10:1.

Preferably, the method according to the invention of preparing nitrate esters is performed in a temperature range of 20 to 50° C., and particularly preferred is a temperature range of 30 to 45° C.

The object of the invention is explained in greater detail by reference to the following examples without thereby being associated with any restrictions:

### EXAMPLE 1

### Preparation of nitroglycerol in a Microreactor

The reaction was performed in a microreactor (or micromixer) composed of the material silicon using the split-and-recombine principle. In this connection, liquid flows are split up and, after traversing various paths, are brought together again. Repeating this flow configuration several times in parallel microchannels results in an efficient mixing of the liquid flows. The microchannel structures of the microreactor are approximately 200 to 300 μm in diameter. The length of the parallel microchannel structures varies between 15 and 20 mm. The educts glycerol and mixed acid (concentrated nitric acid and concentrated sulfuric acid containing 4 wt % oleum in a weight ratio of 1:1) were pumped into the microreactor by means of injection pumps. The microreactor was temperature-controlled in a water bath to 50° C. After leaving the microreactor, the reaction mixture flowed through a 50 cm long, temperature-controlled Teflon capillary as dwell section having an internal diameter of 800 μm. The microreactor and Teflon capillary together form the microreaction system. The volumetric flows of the educts and the dwell time in the microreaction system are specified in Table 1. The raw nitroglycerol product composition obtained was analysed by means of known high-pressure liquid chromatography (HPLC). The results are likewise specified in Table 1.

TABLE 1

| | | | | Product composition/% | | | |
|---|---|---|---|---|---|---|---|
| V_{glycerol}/ | V_{mixed acid}/ | | T_{R}/ | | 1,2- | 1,3- | |
| (ml/min) | (ml/min) | DT/s | ° C. | GMN | GDN | GDN | %-GTN |
| 0.8 | 2.75 | 4.2 | 50 | 0.02 | 0.51 | 2.27 | 91.15 |

V: volumetric flow; DT: dwell time; T_{R}: reaction temperature GMN: glyceryl mononitrate; GDN: 1,2- and 1,3-glyceryl dinitrate; GTN: glyceryl trinitrate

### EXAMPLE 2

### Preparation of nitroglycerol in a System Comprising two Microreactors

The esterification of glycerol was performed analogously to Example 1 with the following differences: Two microreactors of the type described in Example 1 were connected in series. In this case, the reaction mixture leaving the first microreactor was divided over the two inlets of the second microreactor. The reaction temperature was 40° C. in this example. The volumetric flows of the educts, the dwell time and the result obtained in the HFLC analysis are summarized in Table 2.

TABLE 2

| | | | | Product composition/% | | | |
|---|---|---|---|---|---|---|---|
| V_{glycerol,}/ | V_{mixed acid,}/ | | T_{R}/ | | 1,2- | 1,3- | |
| (ml/min) | (ml/min) | DT/s | ° C. | % GMN | GDN | GDN | GTN |
| 0.4 | 1.36 | 8.6 | 40 | 0.01 | 0.44 | 1.93 | 93.25 |

V: volumetric flow; DT: dwell time; T_{R}: reaction temperature GMN: glyceryl mononitrate; GDN: 1,2- and 1,3-glyceryl dinitrate; GTN: glyceryl trinitrate

### EXAMPLE 3

### Preparation of nitroglycerol in a Simple T-Microreactor

Glycerol was esterified in a microreaction system comprising a glass T-piece having an internal channel diameter of 800 μm and an adjoining 50 cm long Teflon capillary. The volumetric flows of the educts, the dwell time and the result obtained in the HPLC analysis are summarized in Table 3.

TABLE 3

| | | | | Product composition/% | | | |
|---|---|---|---|---|---|---|---|
| V_{glycerol}/ | V_{mixed acid}/ | | T_{R}/ | | 1,2- | 1,3- | |
| (ml/min) | (ml/min) | DT/s | ° C. | GMN | GDN | GDN | GTN |
| 0.4 | 1.36 | 8.6 | 40 | 0.02 | 0.52 | 2.09 | 90.83 |

V: volumetric flow; DT: dwell time; T_{R}: reaction temperature GMN: glyceryl mononitrate; GDN: 1,2- and 1,3-glyceryl dinitrate; GTN: glyceryl trinitrate

The results achieved in Examples 1 to 3 were also achieved under the same process conditions with other microstructured reactors based on other mixing principles (for example, multilamination).

1. Method of preparing liquid nitrate esters, characterized in that an alcohol solution and a nitrating acid are mixed in a microreactor.

2. Method according to claim 1, characterized in that the internal channel diameter of the microreactor is at least 50 μm.

3. Method according to claim 1, characterized in that the internal channel diameter of the microreactor is at least 100 μm.

4. Method according to claim 1, characterized in that the internal channel diameter of the microreactor is not more than 3000 μm.

5. Method according to claim 1, characterized in that the internal channel diameter of the microreactor is not more than 1000 μm.

6. Method according to claim 1, characterized in that the flow of the liquids in the microreactor is laminar.

7. Method according to claim 1, characterized in that the flow of the liquids in the microreactor has a Reynolds number of <1000.

8. Method according to claim 1, characterized in that the microreactor contains microstructured passive mixing structures.

9. Method according to claim 1, characterized in that the microreactor contains T- or Y-mixing structures.

10. Method according to claim 1, characterized in that the microreactor contains glass or silicon as material.

11. Method according to claim 1, characterized in that the microreactor contains metal, ceramic or enamel as material.

12. Method according to claim 1, characterized in that the method is performed under isothermal conditions.

13. Method according to claim 1, characterized in the microreactor employs the split-and-recombine principle or the multilamination principle.

14. Method according to claim 1, characterized in that a monohydric or polyhydric alcohol is used as alcohol.

15. Method according to claim 1, characterized in that glycerol is used as alcohol.

16. Method according to claim 1, characterized in that a mixture of concentrated sulfuric acid and concentrated nitric acid in a weight ratio of 0.8:1 to 1.2:1 is used as nitrating acid, wherein the sulfuric acid may in turn contain up to 10 wt % oleum.

17. Method according to claim 1, characterized in that glycerol is used as alcohol and the molar ratio of HNO₃ to glycerol is 3:1 to 10:1.

18. Method according to claim 1 for the preparation of a mono-, di- or polynitrate ester.

19. Method according to claim 1 for the preparation of trinitroglycerol or glycyl dinitrate ester.
